(19) United States
(12) Patent Application Publication
(10) Pub. No.: US 2010/0145435 A1
(43) Pub. Date: Jun. 10, 2010

(54) IMPLANTABLE PROSTHETIC VALVE STENT

(76) Inventors: Valerian Voinov, Toronto (CA); Romul Boldyrev, Jerusalem (IL); Valentin Voinov, Cincinnati, OH (US)

Correspondence Address: MOORE & VAN ALLEN PLLC, P.O. BOX 13706, Research Triangle Park, NC, 27709, US

(21) Appl. No.: 12/664,334

(22) PCT Filed: Feb. 21, 2008
(86) PCT No.: PCT/IB08/00521

§ 371 Date: Dec. 11, 2009

(30) Foreign Application Priority Data
Feb. 21, 2008 (IB) PCT/IB2008/000521

(51) Int. Cl. A61F 2/82

(52) U.S. Cl. 623/1.26; 623/1.24

(57) ABSTRACT

An implantable PROSTHESIS with formed power couch+, including a primary tube as an half finished product in a form of an additional support stent, the additional support stem forms a circular structure upon its expansion that allows to reduce the total mass of the implantable valve device upon keeping of its high dynamics stability and to establish axis-symmetrical shape similar to hyperboloid. The leaflets of a valve device functional part are provided a system of a low-profile guiding spiral relief, under which blood flow imposes additionally a component of a spiral movement.

### FIELD of the INVENTION

The present invention relates to implantable devices. In particular, it relates to a valve prosthesis for cardiac implantation or for implantation in other body cannels.

### BACKGROUND OF THE INVENTION

Many different implantable valve prosthesis devices are known, including those which are used in percutaneous technologies for implantation in body channels. One of such effective technologies for implantation is the system presented in the U.S. Pat. No. 6,730,118 B2 entitled Implantable Prosthetic Valve. Not going into details of this technology and its preparatory stages and structure of a biological part of the tricuspid valve prosthesis device, we should only mention that its basic drawbacks are laying in its power part, i.e. in its metallic structure.

The metallic part of the valve prosthesis device is the support stent in a form of a cylindrical net shell, containing transverse (relative to the cylinder axis) closed sinuous circles, interconnected between them by large number of longitudinal beams. Three of them are executed with holes for connection with the biological part of the valve prosthesis device. Primarily, the metallic construction correlates to the nominal values of mean size aortic heart valve, diameter of 23 mm. According to clinical requirements for implantation of the valve prosthesis device, this diameter can vary from 19 to 25 mm. In the support stent with primary diameter of 23 mm, the thrusting angle for elements of the transverse sinuous circles is 90° that allows to perform crimping of the valve prosthesis device on an inflatable balloon catheter freely.

Upon implantation of the valve prosthesis device the support stent expands again till 23 mm and thrusting angle of elements of the transverse sinuous circles becomes equal to 90°. If the diameter of the valve prosthesis device is increased till 25 mm, then the thrusting angle of the elements of the transverse sinuous circles becomes larger than 90°. If the diameter of the valve prosthesis device is reduced till 19 mm, then the thrusting angle of the elements of the transverse sinuous circles becomes lesser than 90°. Sinuosity of the transverse sinuous circles of the support stent is the reason for low capability to resist external radial loads. The lowest resistibility for radial loads is attributed to valve prosthesis device of 19 mm diameter.

The described drawback is compensated by increase in thickness of the cylindrical support stent. For example, to 0.5 mm and more. However, it does not eliminate the danger of recoil of the support stent, which in addition is subjected to significant wear and tear changes under impulses from contracting heart.

Another drawback of the support stent is its cylindrical form, preventing performance of longitudinal fixation of the valve prosthesis device in implantation site. Valve device is being held in place of implantation only by its thrusting force. Pressure increase in balloon catheter results in increase of diameter of the support stent (possibly over expansion) and thrusting force, but without altering the cylindrical form of the support stent. Such a form also brings the necessity to increase thickness of the support stent. The most rational structure of the support stent is the structure, including of circular circles (thrusting angles of elements of the transverse sinuous circles equal to 180° or close to this value).

In such a case, the picture of elements deformation of the support stent is changed qualitatively, bending deformations are eliminated, elements of the support stent undergo only compressing forces, and change in their form (collapse) can occur only under significant accumulated compressing forces, correlating to the threshold violation of stability condition of the elements of the support stent. Up to this threshold the recoil of the cylindrical support stent with circular circles is absent.

Threshold violation of the stability condition of elements of the support stent is maintained by radial forces, compressing the support stent under cardiac impulses and they are much higher then those causing a fatal recoil of the support stent with transverse sinuous circles.

Using the circular circles in the support stent provides:

a) The necessary radial resistibility of the support stent under much reduced thickness.

b) Favorable form of the support stent provides its longitudinal fixation in implantation site, and also improves hemodynamics in entrance and exit of the valve prosthesis device. Concurrently the support stent can be not of cylindrical form. For example, in the form similar to hyperboloid (rims diameters are larger than in the support stent interior). Hyperboloid formation of the support stent is achieved by varying the length of the circle of the circular circles, because under increase of pressure in the balloon catheter, circles diameters cannot be increased more than preset previously (exclusion of over expansion).

c) Exclusion of wear and tear of elements of the support stent due to absence of cyclical bending deformations.

However the existing technology of the valve prosthesis device contains certain preparatory stages for its implantation (embodiment of a biological part, its set-up, crimping etc.) and it prevents use of circular circles in the support stent. Therefore, along with the suggested innovations, there is full conservation of the structure and technology of Implantable Prosthetic Valve in accordance with the U.S. Pat. No. 6,730,118 B2, except thickness reduction of the support stent. The suggested coalition is being newer, more perfect clinical valve device.

Further on, description of the present invention the Implantable Prosthetic Valve is entitled as an implantable PROSTHESIS.

The present invention provides a series of new concepts in the field of aortic valves and other human ducts.

### SUMMARY OF THE INVENTION

The purpose of the present invention is the creation of an implantable PROSTHESIS with formed power couch, based on a complex of a positive characteristics of the force shell of the circular structure and by that the valve device in accordance with the U.S. Pat. No. 6,730,118 B2 acquires new potential for wider clinical application.

In accordance with a preferred embodiment of the present invention, the implantable PROSTHESIS is suitable for implantation in body ducts, the implantable valve device comprising:

a primary tube as a half finished product (PT) in a form of an additional support stent with transverse sinuous circles, with thrusting angles of ˜90°; and
the (PT) of the additional support stent with the transverse sinuous circles, with thrusting angles of ˜90° is executed with an interior diameter more than an external diameter of the implantable PROSTHESIS after crimping; and
the (PT) of the additional support stent with the transverse sinuous circles, with thrusting angles of ˜90°, with the sum length of all elements for each transverse sinuous circle form circular circle upon expansion till nominal size of implantation in such a way that thrusting angles of elements become equal to 180° or close to that value; and
the (PT) of the additional support stent with the transverse sinuous circles is executed with unequal angles of thrust of elements, whereas for each of the transverse sinuous circles the thrusting angles of elements are reduced from the interior to the rims of the additional support stent in such a way that upon expansion the sequence of circular circles of nominal sizes form an envelope surface similar to hyperboloid; and
a crimping of the (PT) of the additional support stent on the external surface of the crimped implantable PROSTHESIS; and
the (PT) of the additional support stent is executed from the same materials as the support stent of the implantable PROSTHESIS.

Furthermore, in accordance with another preferred embodiment of the present invention, a (PT) of an additional support stent with transverse sinuous circles, with thrusting angles of elements of 90° is executed from tubes of 13.5 mm÷18.0 mm diameters in accordance with nominal sizes of an implantable PROSTHESIS of 19 mm÷25 mm diameters. Furthermore, in accordance with another preferred embodiment of the present invention, a (PT) of an additional support stent with transverse sinuous circles, with thrusting angles of elements of 90°, with the nominal size of the implantable PROSTHESIS of 23 mm diameter is executed from the tube of 16.5 mm diameter in accordance with the drawing of the support stent of the implantable valve device in the scale of 0.71.

Furthermore, in accordance with another preferred embodiment of the present invention, a (PT) of an additional support stent with transverse sinuous circles, with thrusting angles of elements of 90°, with the nominal size of the implantable PROSTHESIS of 19 mm diameter is executed from the tube of 13.5 mm diameter in accordance with the drawing of the support stent of the implantable valve device in the scale of 0.71.

Furthermore, in accordance with another preferred embodiment of the present invention, a (PT) of an additional support stent with transverse sinuous circles, with thrusting angles of elements of 90°, with the nominal size of the implantable PROSTHESIS of 25 mm diameter is executed from the tube of 18 mm diameter in accordance with the drawing of the support stent of the implantable valve device in the scale of 0.72.

Furthermore, in accordance with another preferred embodiment of the present invention, a (PT) of an additional support stent is executed from tubes of 8 mm÷20 mm diameters, whereas thrusting angles of the elements of transverse sinuous circles are chosen in such a way that upon expansion till nominal size of implantation the transverse sinuous circles form circular structure with thrusting angles of elements of 180°÷170°.

Furthermore, in accordance with another preferred embodiment of the present invention, a (PT) of an additional support stent is executed with minimal possible virtual thickness of the cylinder, resistance of which sustains a thrusting force of a balloon catheter. Furthermore, in accordance with another preferred embodiment of the present invention, a (PT) of an additional support stent is executed with ˜0.05 mm÷0.10 mm of virtual thickness for connection with an implantable PROSTHESIS with ˜0.25 mm virtual thickness of the cylinder of the support stent.

Furthermore, in accordance with another preferred embodiment of the present invention, a (PT) of an additional support stent is executed with increased number of transverse sinuous circles.

Furthermore, in accordance with another preferred embodiment of the present invention, a (PT) of an additional support stent is executed with increased number of longitudinal beams.

Furthermore, in accordance with another preferred embodiment of the present invention, a (PT) of an additional support stent is executed with reduced number of longitudinal beams.

Furthermore, in accordance with another preferred embodiment of the present invention, a (PT) of additional support stent, included usual circles or pair of strengthened circles, is executed with two continuous beams, situated opposite to a stent length in one plane, whereas elements of each continuous beam between two neighboring circles are presented in a form of the bars of equal resistance to bending along them cylindrical surface.

Furthermore, in accordance with another preferred embodiment of the present invention, a circular structure of a (PT) of an additional support stent is formed without limitation of a size of the circles diameters.

Furthermore, in accordance with another preferred embodiment of the present invention, a (PT) of an additional support stent is executed with disconnected circles, combined along a stent length in one spiral line with one or several threads, whereas instead of two continuous beams, situated opposite to the stent length in one plane, preserve one only. Furthermore, in accordance with another preferred embodiment of the present invention, an additional support stent is executed with length longer than length of the support stent of the implantable PROSTHESIS, whereas the length of the additional support stent is increased in a way that one of its parts is inside the descending aorta.

Furthermore, in accordance with another preferred embodiment of the present invention, an additional support stent is executed with length according to the size of the annulus of aortic heart valve for later precise positioning of the implantable valve device.

Furthermore, in accordance with another preferred embodiment of the present invention, a (PT) of an additional support stent is executed from Nitinol (nickel titanium).

Furthermore, in accordance with another preferred embodiment of the present invention, a (PT) of an additional support stent is provided with heavy metal markets so as enable tracking and determining the valve device position.

Furthermore, in accordance with another preferred embodiment of the present invention, a (PT) of an additional support stent is made in an individual form for various clinical applications.

Furthermore, in accordance with another preferred embodiment of the present invention, a (PT) of an additional support stent is made in an individual form as an annuloplasty thin-walled ring for repair of the aortic heart valve damaged by acquired or congenital disease.

Furthermore, in accordance with another preferred embodiment of the present invention, an additional support stent, made in an individual form, is provided with a drug structure, whereas the drug structure is located along spiral line on the stent interior surface.

Furthermore, in accordance with another preferred embodiment of the present invention, there is provision of a method of approaching the aortic valve from the descending aorta for deploying an implantable PROSTHESIS combined with an additional support stent at the natural aortic valve position at the entrance to the left ventricle of a myocardium of a patient, the method comprising the steps of:

a) guiding a balloon catheter having a proximal end and a distal end, having first and second independently inflatable portions, the first inflatable portion located at the distal end of the catheter and the second inflatable portion located adjacently behind the first inflatable portion, through a patient's aorta using a guiding tool, a deployable additional support stent being mounted over on the first inflatable portion of the balloon catheter and a deployable implantable PROSTHESIS being mounted over the second inflatable portion of the balloon catheter, the deployable implantable additional support stent and deployable implantable PROSTHESIS being kept at a determined distant apart until the first inflatable portion of the balloon catheter is positioned the deployable additional support stent at the natural aortic valve position; and
b) inflating the first inflatable portion of the balloon catheter so as to deploy the additional support stent, as less sensitive for longitudinal placing, in position at the natural aortic valve position; and
c) deflating the first inflatable portion of the balloon catheter; and
d) guiding the balloon catheter until the second inflatable position of the balloon catheter is positioned at the natural aortic valve position; and
e) inflating the first inflatable position of the balloon catheter into the left ventricle thus anchoring the deployable implantable PROSTHESIS in position and substantially block blood flow; and
f) inflating the second inflatable position of the balloon catheter so as to deploy the implantable PROSTHESIS in position at the natural aortic valve position; and
g) deflating the first and second inflatable positions of the balloon catheter; and
h) withdrawing the balloon catheter from the patient's body.

Furthermore, in accordance with another preferred embodiment of the present invention, there is provision of the same method for deploying an implantable PROSTHESIS combined with an additional support stent at the natural aortic valve position, whereas approaching of the aortic valve position executes from the left ventricle of a myocardium of a patient after performing a trans-septal puncture.

Furthermore, in accordance with another preferred embodiment of the present invention, there is provided a method of approaching the aortic valve from the descending aorta for deploying an implantable PROSTHESIS combined with an additional support stent at the natural aortic valve position at the entrance to the left ventricle of a myocardium of a patient, the method comprising the steps:

a) guiding a balloon catheter having a proximal end and distal end, having first and second independently inflatable portions, the first inflatable portion located at the distal end of the catheter and the second portion located adjacently behind the first inflatable portion, though a patient's aorta using a guiding tool, a deployable additional support stent combined with annular stent device being mounted behind the implantable additional support stent position and all of this being mounted over the first inflatable portion of the balloon catheter and a deployable implantable PROSTHESIS being mounted over the second inflatable portion of the balloon catheter, the deployable additional support stent combined with the predetermined apart annular stent device and deployable implantable PROSTHESIS being kept at a predetermined distant apart until the first inflatable portion of the balloon catheter is positioned the deployable additional support stent, at the natural aortic valve position; and
b) inflating the first inflatable portion of the balloon catheter so as to deploy the additional support stent combined with the pretermined apart annular stent device, as less sensitive for longitudinal placing, in position at the natural aortic valve position; and
c) deflating the first inflatable portion of the balloon catheter; and
d) guiding the balloon catheter until the second inflatable position of the balloon catheter is positioned at the natural aortic valve position; and
e) inflating the first inflatable position of the balloon catheter into the left ventricle thus anchoring the deployable implantable PROSTHESIS in position and substantially block blood flow; and
f) inflating the second inflatable position of the balloon catheter so as to deploy the implantable PROSTHESIS in position at the natural aortic valve position; and
g) deflating the first and second inflatable positions of the balloon catheter; and
h) withdrawing the balloon catheter from the patient's body.

Furthermore, in accordance with another preferred embodiment of the present invention, there is a provision of a method for percutaneous deployment of an implantable PROSTHESIS with formed power couch with use of the BYRASS system, under which in the beginning there is setting of an additional support stent at the natural aortic valve position, after which the implantable valve device is implanted in the same position according to known stages of this procedure.

Furthermore, in accordance with another preferred embodiment of the present invention, there is provided a method of cyclical displacing of an inflated balloon catheter at a short distant in implantation site in a structure of a clinical procedure, the method comprising the steps:

a) an inflate of the balloon catheter till the level of lumen closing in implantation site; and
b) a deflate of the balloon catheter, providing its sparing repositioning in the proper direction in implantation site; and
c) repetition of inflate-deflate-displacing cycles with frequency of an approximate to frequency and multiple to frequency of cardiac contractions; and
d) beginning of inflate cycle in cycles of inflate-deflate-displacing is performed parallel to beginning of diastole; and
e) cycles of the inflate-deflate-displacing continue to perform until there is achievement of new exactly preset state of the balloon catheter in implantation site.

Furthermore, in accordance with another preferred embodiment of the present invention, there is provided an automatic device for cyclical displacing of an inflated balloon catheter at a short distant in implantation site in a structure of a clinical procedure, the automatic device can execute cycles of inflated-deflated of the inflated balloon catheter with frequency in accordance with cardiac contractions frequency so that beginning of inflated-deflated of the inflated balloon catheter correlates with the beginning of the diastole.

Furthermore, in accordance with another preferred embodiment of the present invention, there is a provision of a method for accompanied correction of blood flow in implantation site of an implantable PROSTHESIS, under which blood flow imposes additionally a component of spiral movement by way of a corresponding low-profile guiding spiral relief on the surfaces of the valve device turned to the blood flow.

Furthermore, in accordance with another preferred embodiment of the present invention, there is a provision of a method for accompanied correction of blood flow in implantation site of an implantable PROSTHESIS, which is made in a form of a stentless aortic biological valve device and contained a cylindrical frame with placed in the leaflets from porcine tissue, whereas on the surface of the leaflets, seen of an observer upon closed of the valve device on the side of the left ventricle, is executed a low-profile guiding spiral relief.

Furthermore, in accordance with another preferred embodiment of the present invention, there is provision of a method for accompanied correction of blood flow in implantation site of an implantable PROSTHESIS, which is made in a form of a stentless aortic biological valve device and contained a cylindrical frame with placed in the leaflets from porcine tissue, whereas an entrance part of the cylindrical frame on the side of the left ventricle is provided a low-profile guiding spiral relief.

Furthermore, in accordance with another preferred embodiment of the present invention, there is provision of a method for accompanied correction of blood flow in implantation site of an implantable PROSTHESIS, which is made in a form of a metallic valve device and contained a cylindrical frame with placed in two flat turn-leaflets together with their spindles of hinges and their saddles, whereas the spindles of the hinges are crossed relatively to each other in a “X” form with according position of the saddles of the flat turn-leaflets, and a crossing-line angle of the spindles of the hinges defines in the field of 10°÷60° magnitude.

Furthermore, in accordance with another preferred embodiment of the present invention, there is a provision of a method for accompanied correction of blood flow in implantation site of an implantable PROSTHESIS, which is made in a form of a corrugated graft and contained transverse corrugations, located along guiding spiral line.

Furthermore, in accordance with another preferred embodiment of the present invention, there is provision of a method for accompanied correction of blood flow in implantation site of an implantable PROSTHESIS, which is made in a form of a corrugated graft and contained transverse corrugations, located along guiding spiral line, whereas created guiding spiral lines of the corrugated graft performed as multiple-threads with tilting angle of guiding spiral line of ˜45° order.

Furthermore, in accordance with another preferred embodiment of the present invention, there is a provision of a method for accompanied correction of blood flow in implantation site of an implantable PROSTHESIS, which is made in a form of a corrugated graft of out of round cross-section, equivalent to the square of device of the round cross-section. Furthermore, in accordance with another preferred embodiment of the present invention, there is a provision of a method for accompanied correction of blood flow in implantation site of an implantable PROSTHESIS, which is made in a form of a corrugated graft of out of round cross-section, equivalent to the square of device of the round cross-section, whereas during the final stage of manufacturing of the graft, it is spiraled and fixated in a way that the created guiding spiral line of ledges of the graft have tilting angle no more than 75°.

Furthermore, in accordance with another preferred embodiment of the present invention, there is a provision of a method for accompanied correction of blood flow in implantation site of an implantable PROSTHESIS, which is made in a form of a corrugated graft of a polygonal cross-section, equivalent to the square of device of the round cross-section. Furthermore, in accordance with another preferred embodiment of the present invention, there is a provision of a method for accompanied correction of blood flow in implantation site of an implantable PROSTHESIS, which is made in a form of a corrugated graft of a polygonal cross-section, equivalent to the square of device of the round cross-section, whereas during the final stage of manufacturing of the graft, it is spiraled and fixated in a way that created guiding spiral line of ledges of the graft has tilting angle no more than 75°.

### BRIEF DESCRIPTION OF THE FIGURES

To understand better the present invention and appreciate its practical applications the following Figures are provided and reference hereafter. It should be noted that the Figures are given as examples only and in no way limit the scope of the invention as defined in the appended claims.

FIG. 1 illustrates an implantable tricuspid valve prosthesis device for percutaneous deployment using a stent, in its deployed-inflated position in accordance with the U.S. Pat. No. 6,730,118 B2 entitled implantable PROSTHESIS under description of the present invention.

FIG. 2 illustrates a schematic view of an implantable PROSTHESIS according to FIG. 1.

FIG. 3 illustrates schematically an evolvent on the plane of its cylindrical surface of a support stent of the implantable PROSTHESIS according to FIG. 1.

FIG. 4 demonstrates schematically an evolvent on the plane of its cylindrical surface of a (PT) of an additional support stent, according to the present invention.

FIG. 5 demonstrates a schematic view of a (PT) of an additional support stent, an evolvent on the plane of its cylindrical surface of which the corresponding to FIG. 4, according to the present invention.

FIG. 6 illustrates schematically a view from one side of a final expanded additional support stent, according to the present invention.

FIGS. 7a, 7b, 7c and 7d demonstrate positions of combining of an implantable PROSTHESIS and an additional support stent and also their final expanded position, according to the present invention.

FIG. 8 demonstrates a scheme of load of a transverse sinuous circle for numerical evaluation of a stent virtual thickness, according to the present invention.

FIG. 9 demonstrates schematically a view by A arrow of FIG. 1 on a leaflet of a tricuspid valve biological part with a low-profile guiding spiral relief of its surface, according to the present invention.

FIG. 10 demonstrates schematically a top view on a metallic valve device, in the cylindrical frame of which is placed two flat turn-leaflets together with their spindles of hinges and their saddles (the closed stage of the flat turn-leaflets), according to the present invention.

FIGS. 11 and 12 demonstrate schematically A-A and B-B cross-sections of the metallic valve device according to FIG. 10, from which the spindles of the hinges are crossed relatively to each other in a “X” form and placed with according position of the saddles of the flat turn-leaflets, according to the present invention.

δ—a crossing-line angle of the spindles of the hinges of the flat turn-leaflets;

—the direction of the spiral movement of blood flow is schematically marked by puncture arrows.

FIG. 13 demonstrates an outward view of a metallic valve device, from which spindles of hinges are crossed relatively to each other in a “X” form and placed with according position of the saddles of the flat turn-leaflets, according to the present invention.

—the central axes of two twisted blood flows, flowing from the valve device to the aorta, are schematically marked by puncture arrows.

### DETAILED DESCRIPTION OF THE INVENTION

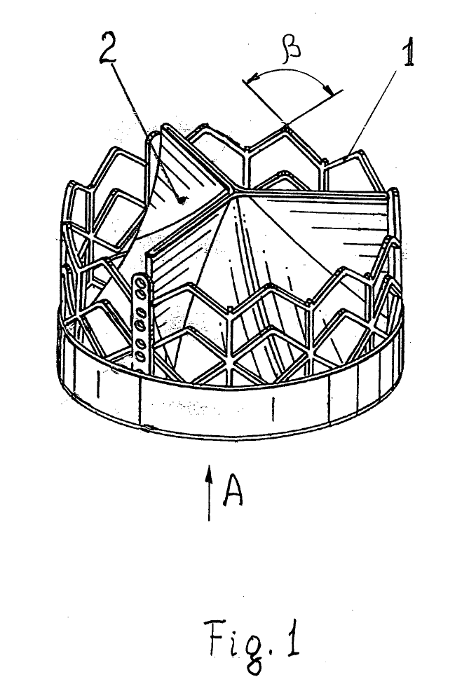

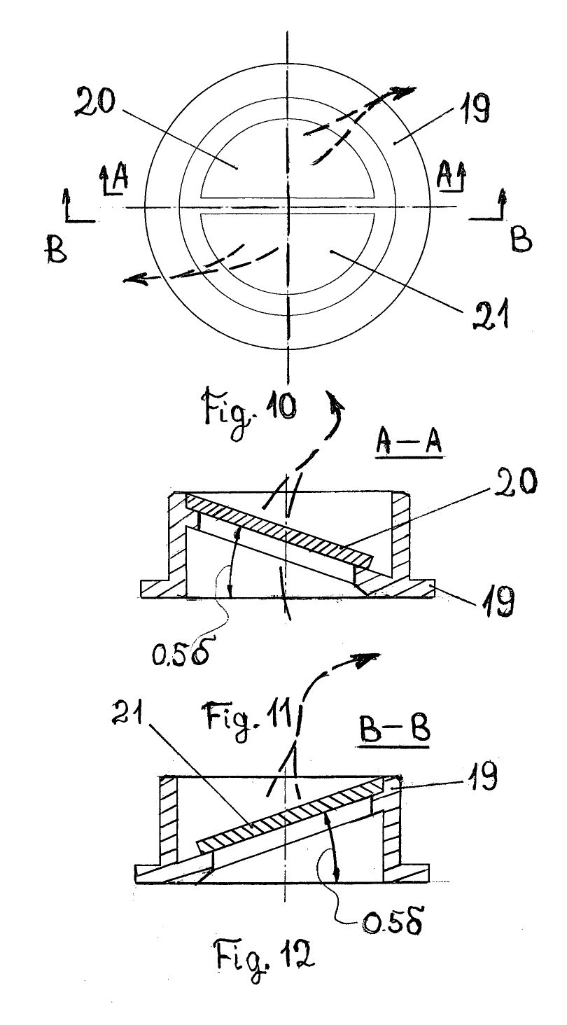

FIG. 1 illustrates a general view of an implantable PROSTHESIS according to the U.S. Pat. No. 6,730,118 B2. Pos. 1 marks a metallic support stent, in which β—thrusting angle of elements of transverse sinuous circles. Pos. 2 marks a biological part of the implantable PROSTHESIS. The A arrow shows on a leaflet of the implantable PROSTHESIS. In particular, on one of the tricuspid biological leaflet 2 (it is demonstrated in FIG. 9). The implantable PROSTHESIS is illustrated in an expanded position accordingly to its initial manufacturing, and also with its final expanded position for implantation. The external diameter of the implantable PROSTHESIS equals 23 mm, thrusting angle (β) equals 90°.

The tube of this very diameter is used to manufacture support stent 1. However upon implantation, the implantable PROSTHESIS can expand from the crimping condition to diameters 19 mm÷25 mm according to its clinical application. Under diameters lesser than 23 mm, the thrusting angle (β) is lesser than 90°. Under diameters larger than 23 mm, the thrusting angle (β) is larger than 90°. Reduction in the thrusting angle leads to significant reduction in resistibility of the implantable PROSTHESIS to radial loads from the cardiac muscle.

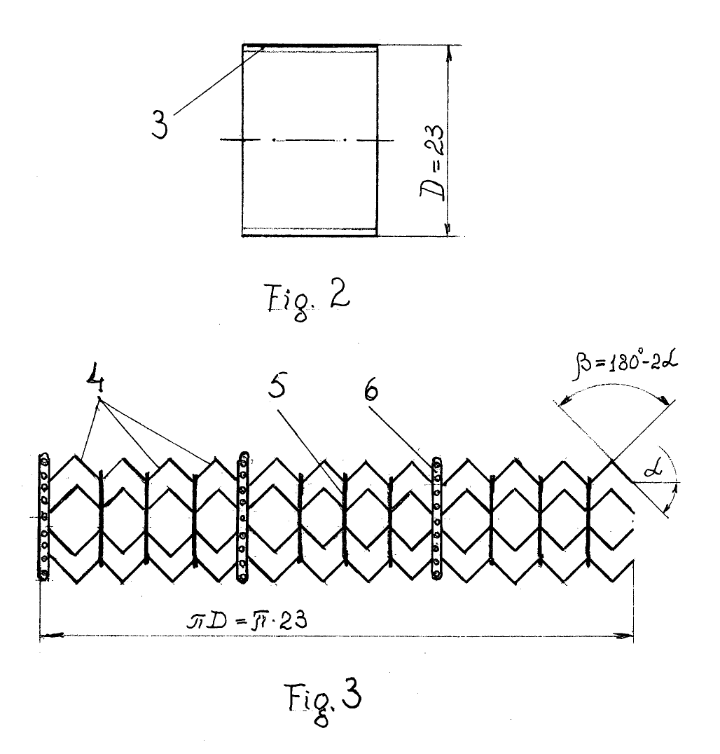

FIG. 2 illustrates a schematic view of an implantable PROSTHESIS according to FIG. 1.

FIG. 3 illustrates schematically an evolvent on the plane of its cylindrical surface of a support stent 1 of an implantable PROSTHESIS according to FIG. 1.

Pos. 4 is marked one from transverse sinuous circles, pos. 5 is marked one from longitudinal beams, pos. 6 is marked one from more wide longitudinal beams with holes for connection with biological part of the implantable PROSTHESIS.

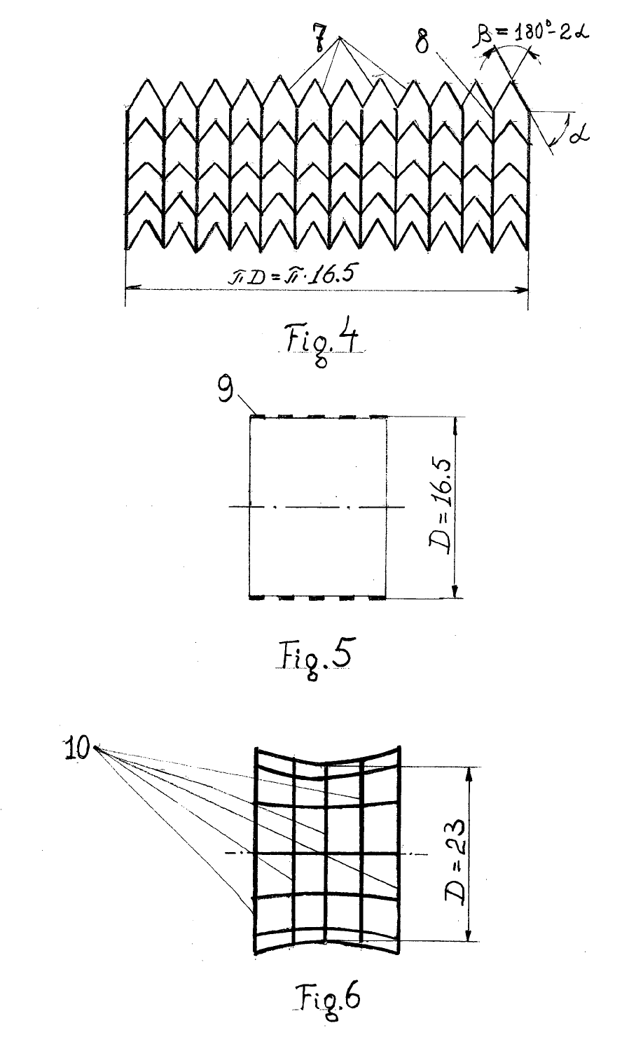

FIG. 4 demonstrates schematically an evolvent on the plane of its cylindrical surface of a (PT) of an additional support stent, according to the present invention.

Pos. 7 is marked one from transverse circles and pos. 8 is marked one from longitudinal beams. The angle values (which can be others by value) are marked with same symbols as on FIG. 3. This also regards to diameter marking.

Here (FIG. 4) demonstrated the evolvent on the plane of the cylindrical surface of the additional support stent of 16.5 mm diameter with thrusting angle of the middle transverse circle of β=90°. Thrusting angles of the transverse sinuous circles, located from the interior to the rims of the evolvent gradually reduced.

FIG. 5 demonstrates a schematic view of a (PT) of an additional support stent 9, an evolvent on the plane of its cylindrical surface of which was illustrated in FIG. 4, according to the present invention.

FIG. 6 demonstrates schematically a view on one side of a final expanded additional support stent, according to the present invention. It is seen that upon expansion transverse sinuous circles transform into circular circles 10 (β˜180°, and middle circular circle has the same diameter as the implantable PROSTHESIS (23 mm), and diameters of the circular circles, located at the rims of the additional support stent, gradually increased accordingly to the reduction in thrusting angles as it was seen to explanation to FIG. 4.

It is important to emphasize that further expansion of circular circles (without their destruction) by any expanding devices is impossible and it basically excludes possibility of their over expansion. At the same time it is important to note that significant increase in resistibility of circular circles to radial load is taking place in range of thrusting angles β=170°÷180° and this can be used if change in diameter of circular circles under ˜2 mm is needed, by altering pressure in the balloon catheter.

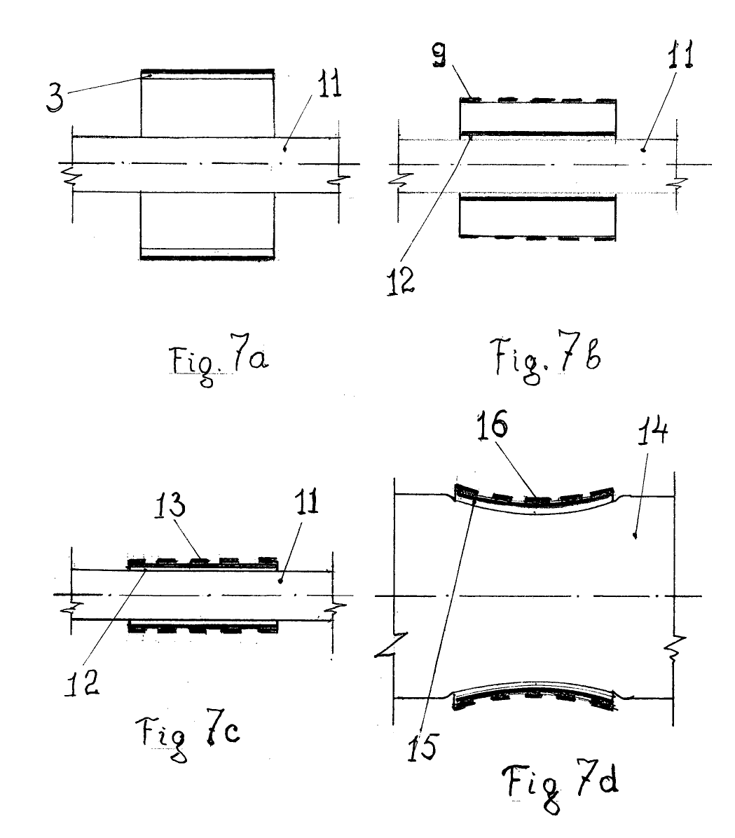

FIGS. 7a, 7b and 7c demonstrate positions of combining of an implantable PROSTHESIS and a (PT) of an additional support stent, according to the present invention.

FIG. 7a demonstrates an implantable PROSTHESIS (pos. 3) being mounted over a balloon catheter, according to the present invention.

FIG. 7b demonstrates a (PT) of an additional support stent (pos. 9) being mounted over a crimped implantable PROSTHESIS (pos. 12) on a balloon catheter (pos. 11), according to the present invention.

FIG. 7c demonstrates a crimped additional support stent (pos. 13) being mounted over a crimped implantable PROSTHESIS (pos. 12) with a balloon catheter (pos. 11), according to the present invention.

FIG. 7d demonstrates a final expanded position of an implantable PROSTHESIS and an additional support stent, according to the present invention. Pos. 14 is marked a balloon catheter. Pos. 15 is marked an expanded implantable PROSTHESIS. Pos. 16 is marked an expanded additional support stent (according to FIG. 6). According to commentary to FIG. 6 over expansion of the implantable PROSTHESIS is excluded, which takes the form of border state of the additional support stent.

From FIG. 7d it is also concluded that by varying the thrusting angles of elements of the additional support stent it is possible to achieve any preset final axial shape of the implantable PROSTHESIS if it is connected with the additional support stent. If the additional stent is executed from Nitinol (it is not illustrated in FIG. 7) then it can be expanded the first. As a result, the expanded additional support stent forms a power couch with high resistibility to the radial load and with preset final axis-longitudinal shape. Later on the expanded implantable PROSTHESIS repeats the shape of the additional support stent similar to hyperboloid and in this case over expansion is excluded and longitudinal form of the implantable valve device prevents axial migration and at the same time is being the optimal shape in regards to blood flow.

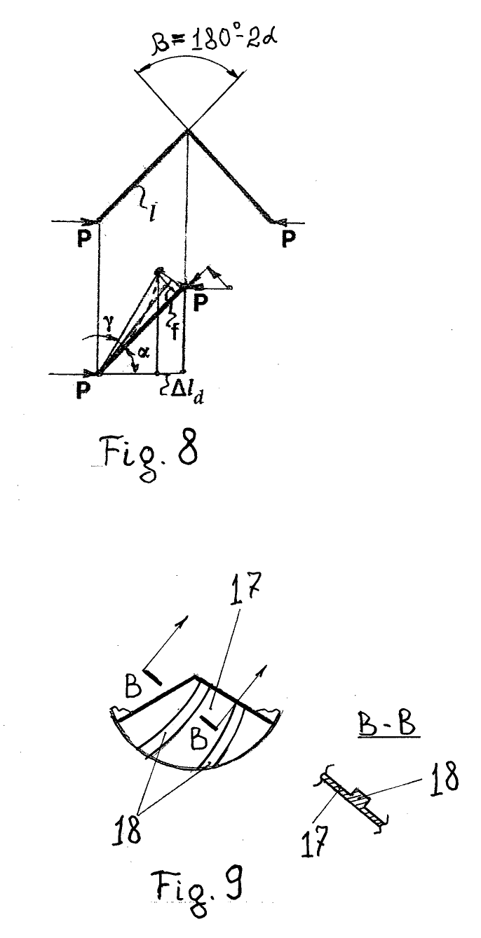

FIG. 8 demonstrates two elements of a transverse sinuous circle with thrusting angle β=180°−2α, on which force P is acting, equivalent to the compressing radial load of the cylindrical shell in the form of a stent. Flexible shells, subjected to external radial loads, could be presented in the form of frames from circular or sinuous circles. Although the effectiveness of resistance of the circular circles to the radial load is obviously higher, it would be advisable to have at least approximate numerical evaluation of the level of such effectiveness. Here it is necessary to make a comparison between two different mechanical systems of resistibility to the radial load.

Upon the radial load, the circular circle, as an arch structure, does not undergo deformations till the condition of collapse (compression of the circle's material is excluded). In the sinuous circle, its structural elements are being deformed immediately, proportionally to the load applied to the sinuous circle.

The radial load on the flexible shell and the frame tends to decrease its geometrical sizes in the condition of complicated tensions scheme. However, in order to numerically compare, it is enough to consider equivalent P force, acting on the comprising elements of the circles in their projections on the plane (FIG. 8).

The elements of the circular circle undergo deformation from material compression only, till the value of P_{cr} critical force, violating the stability of the element.

The elements of the sinuous circle undergo deformation of bending termed f (the bending of the circle's element, proportional to the radial load), and the flexible shell itself decreases upon the diameter as a result of recoil, which value is limited by the work condition of the flexible shell.

Let us turn to the known formulas:
For the element of the sinuous circle

$$f = \frac{1}{3} \cdot \frac{P \cdot sin α \cdot {l}^{3}}{EJ} , \qquad ( 1 )$$

For the element of the circular circle

$${P}_{cr} = k \cdot \frac{EJ}{{( 2 l \cdot cos α )}^{2}} = \frac{EJ}{{l}^{2}} \cdot {( \frac{π}{cos α} )}^{2} , \qquad ( 2 )$$

where E—modulus of elasticity of the circle material; J—moment of inertia of the circle's element cross-section; l—length of the sinuous circle element; k—coefficient, depending on rod's ends closing up (here k=4π²); α—angle of element's incline of the sinuous circle.

Let us set the value of a relative bending of the element of the sinuous circle

$${f}_{o} = \frac{f}{l} ,$$

and will set the value, under which fₒ reaches, if P_{cr} critical force of the rectilinear element is inserted to formula (1), characteristic for sinuous circle, P=P_{cr}

$${f}_{o} = \frac{f}{l} = \frac{EJ}{{l}^{2}} \cdot {( \frac{π}{cos α} )}^{2} \cdot \frac{1}{3} \cdot \frac{sin α \cdot {l}^{2}}{EJ} ; {f}_{o} = \frac{{π}^{2}}{3} \cdot \frac{tg α}{cos α} , \qquad ( 3 )$$

Value of fₒ bending is large enough in order to be accepted as real, but at the same time this conditional parameter allows to evaluate co-relation of R, ratio of critical loads for circular and sinuous circles co-relation of R, ratio of critical loads, is defined by direct ratio of equalities (3) and (5) under critically defined value of Ω recoil.

For the frame from sinuous circles the value of the Ω recoil will be

$$Ω = \frac{Δ D}{D} = \frac{Δ {l}_{d}}{{l}_{d}} ,$$

where ΔD—decline of D diameter of the frame; Δl_{d}—decline of element's length upon bending, measured along the circumference of the frame, Δl_{d}=l·cos α.

It is following from FIG. 8

$$\begin{matrix}Ω = \frac{Δ {l}_{d}}{{l}_{d}} \\ = \frac{1}{l \cdot cos α} ( f \cdot sin α + f \cdot tg \frac{γ}{2} \cdot cos α ) \\ = \frac{f}{l} \cdot ( tg α + tg \frac{γ}{2} ) \approx ( tg α + tg \frac{γ}{2} ) \cdot sin γ ,\end{matrix}$$

where γ—angle, corresponding to the bending of l sinuous circle element.

With small bending of the circle's element (γ<10°) it is reasonable to consider

$$\frac{f}{l} = {f}_{o} \approx sin γ , \qquad ( 5 )$$

As an example let us present the correlation of working characteristics of the circular and sinuous circles under α=45° (see the Table).

| | γ | | | | |
|---|---|---|---|---|---|
| | 2° | 4° | 6° | 8° | 10° |
| Ω | 0.0355 | 0.0722 | 0.11 | 0.1489 | 0.1888 |
| R | 133.3 | 66.7 | 44.5 | 33.4 | 26.8 |

It is seen from the table that with radial load causing recoil of the sinuous circle around 11%, the circular circle is able to stand the radial load without collapse larger in 44.5 times. Also it is necessary to emphasize that with decrease in angle of α, when the shape of the sinuous circle approaches to the circular shape, the effectiveness of resistance to radial load of the sinuous circle increases, but nevertheless, does not reach the level of circular circle resistance.

Based on conditions of use of the implantable PROSTHESIS let us assume that it's fatal recoil, preventing axial migration, is 6%. Then forces rate, causing collapse of a circular circle and 6% recoil of a sinuous circle can roughly be equal to 70 (see Table). Based on that it is concluded that the necessary and checked thickness of support stent of the implantable PROSTHESIS (really accepted in structure of 0.5 mm) can be reduced in 70 times under condition that circular circles are used in the construction. So in the present invention the additional support stent with the thickness of [0.5 mm: 70]=0.007 mm could resolve force drawbacks of the implantable PROSTHESIS with sufficient reserve. However, taking into consideration deviations and known differences between theory and practice, we will accept coefficient of reliability with a magnitude of 7 (!) and minimal (technically performed) thickness of the additional support stent, equal to ˜0.05 mm. In addition, with large enough diameter of the implantable PROSTHESIS (23 mm) it is expedient to somewhat increase the thickness of the additional support stent and decrease the thickness of the implantable valve device.

In accordance with the present invention it is recommended to accept the thickness of the support stent of the implantable PROSTHESIS in range of ˜0.25 mm and the thickness of the additional support stent in range of ˜0.05 mm÷0.10 mm.

FIG. 9 demonstrates a view by A arrow of FIG. 1 on a leaflet 17 of a tricuspid valve biological part with a low-profile guiding spiral relief 18 of the surface according to the present invention. The B-B cross-section in FIG. 9 demonstrates a schematic profile of the guiding spiral relief 18, which can be executed in different shapes and sizes, including according to the shape of material fold of the valve biological part. The low-profile guiding spiral relief (pos. 18) is executed only along visible part of the surface along

A arrow of the leaflet 17, in order to prevent closure of the implantable PROSTHESIS. The low-profile guiding spiral relief 18 can be executed separately as well, in form of guides, predominantly on elongated entrance part of the implantable PROSTHESIS. The function of the spiral guides (pos. 18) of the flow may be expanded. In valve devices with leaflets from organic tissue, the guides' system can form a flexible “corset” from tissue, assisting to a reduction in time of closing of a valve device. In metallic valve devices the guides form ribs of stiffness on the surface of the leaflets, permitting a reduction of leaflets mass without change in their stability. For both variants of the guides, as the reason for forming of the spiral flow, there is an exclusion of a generation of stagnant zones of blood flow on the surfaces of a valve device due to better wash away of these surfaces.

Another example of the organization of spiral movement of blood flow in an implantation site, using metallic valve device. The valve device contains a cylindrical frame and in it there are two flat turn-leaflets together with their spindles of hinges and their saddles, but the spindles of the hinges of the flat turn-leaflets set in the plane of the longitudinal axis of the cylindrical frame.

FIG. 10 demonstrates schematically a top view on a reconstructed metallic valve device, but FIGS. 11, 12-A-A and B-B cross-sections according to FIG. 10.

Two flat turn-leaflets 20, 21 are placed in the cylindrical frame 19, whereas their spindles of the hinges are crossed relatively to each other in a “X” form with a crossing-line angle of δ in according with position of the saddles of the flat turn-leaflets. Each flat turn-leaflet in bent towards the position plain of the valve device to 0.5δ angle, the value of which defines in the field of 5°÷30° magnitude.

FIG. 10 demonstrates the reconstructed valve device upon the closed stage of the flat turn-leaflets 20, 21. In the open stage the flat turn-leaflets 20, 21 are turned to 90° angle and are connecting by their outer surfaces. The blood flow, marked schematically on FIGS. 11, 12 by puncture arrows, twists in spiral line upon exit from the valve device to the aorta. Together with that, there are two separated and twisted blood flows, flowing in opposite directions, are being combined upon exit from the valve device and form a common spiral flow.

The cylindrical frame 19 of the metallic valve device may be modified according to the direction of the spiral blood flow, exiting from the left ventricle to the aorta.

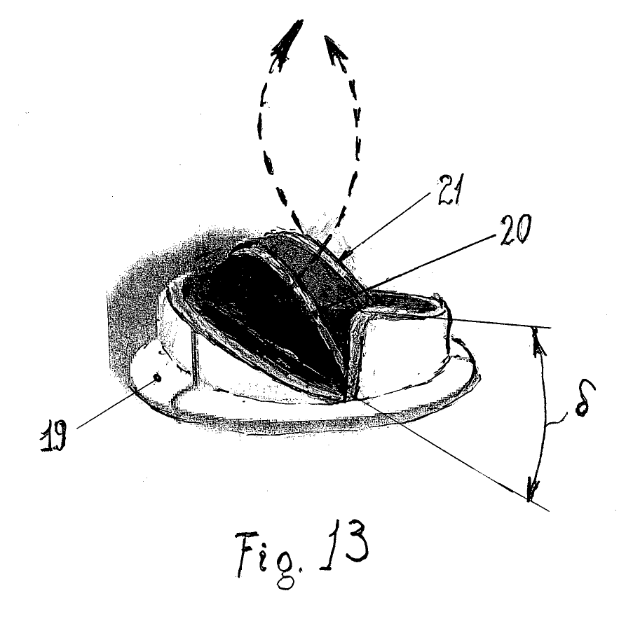

FIG. 13 demonstrates an outward view of a metallic valve device, from which spindles of hinges are crossed relatively to each other in a “X” form and placed with according position of the saddles of the flat turn-leaflets (pos. 20, 21). Here the central axes of two twisted blood flows, flowing from the valve device to the aorta, are schematically marked by puncture arrows.

Additional spiral movement of blood flow upon exit from the left ventricle (cavity with larger cross-section) to the aorta (cavity with smaller cross-section) facilitate the process of blood ejection from the left ventricle to the aorta during systole. This defines the conditions necessary for creation blood volume pressure in the left ventricle with less exertion by myocardial muscle. By that it is possible to decrease heart volume sizes until normal, nominal values.

Except own increase in velocity vector upon spiral movement of blood flow one positive effect is created. Rotating component of blood flow possesses centrifugal force which compresses thin layer of the flow adjacent to the vessel wall and having almost zero velocity. The movement of the isolated compressed thin layer is happening to the side of the lesser pressure where spiral movement and centrifugal forces related to it are absent. The flow of the isolated compressed thin layer to the site opposite to the common movement of blood flow directs its major fractions (possible coagulations) to the centre of the flow. Such contra-flow of the compressed thin layer decreases hydraulic friction of blood flow upon vessel wall, easing its flow through the implanted prosthesis and thus creating more sparing conditions for the normal functioning of a reconstructed aortic heart valve. It is assumed that hydraulic friction of blood flow on the peripheral vessel wall is being decreased along all the surface of the organized spiral movement of blood flow.

For visual explanation of the described process of layered fluid flow it is enough to mix intensively tea with tea-leafs in the transparent glass. When the spoon is removed and spiral movement of the fluid is still present, the tea-leafs, which are heaver than fluid, subside in a form of a hillock with sharp edge on the bottom of the glass and locate on its axis. At the same time, the tea-leafs, which are lighter than fluid, subside on its surface in a form of a cone with a top directed downwards along the axis of the glass.

For both groups of tea-leafs are seemingly not influenced by centrifugal force, which should move them towards the walls of the glass. However, the centrifugal force is acting, but unable to overcome the flow of compressed fluid layer, pressed out from the walls of the glass and forming two circles (torus) of circulation, collected the tea-leafs on the axis of the glass.

The mentioned compressed thin fluid layer in the blood flow also functions as a lubricant for the main flow and by that eases its transitions upon changing and sharply changing cross-sections of a blood vessel because spiral movement destroys stagnated zones, slowing the flow, and makes the flow more regulated.

The concept of processes of liquid friction remind to some degree the elements of the theory of dry friction. In this case it is appropriately to address one of the known problems of N. E. Zukovsky relating the reduction of friction forces in one priority shifts. In particular, upon spiral movement. The priority shift of the spiral movement is the shift along the axis of a screw and it is only part (component) from complete shift, directed by tangent to the spiral line under influence of the full friction force, defined by the process. Naturally, in the priority shift (along the axis of a screw) the friction force will be reduced together with reduced from complete shift of its component.

At the everyday level the problem of N. E. Zukovsky can be illustrated by example of an extraction of a tightly pressed cork from a bottle, when cork not only is being pulled out from the bottle, but at the same time is being turned around. This assists the procedure of extraction.

This very maneuver of the more sparing movement of blood flow may be used technologically not only for the field of prosthesis of the aortic heart valve, but also for different modifications of aortic bioprosthesis' (including aortic bypass grafts) and for cardiovascular current stents that improves compatibility of natural tissue with implantant, and it is always possible to create elements of a spiral movement in prosthesis with the assistance of special corrugation or giving it a certain shape.

Thus, realization of the present invention provides considerable increase in resistibility of the implantable PROSTHESIS to radial forces impacted by contracting heart muscle, reaching its necessary value with preset rigidity reserve and with reduction in total mass of the implantable valve device.

A possibility for formation of axis-symmetrical shape of a metallic part of the implantable PROSTHESIS has been created in order to prevent its axial migration and achievement of more favorable blood flow from the left ventricle to the aorta.

The wear and tear of the implantable PROSTHESIS cover impacted by the forces of contracting heart muscle has been excluded.

The above advantages take place with complete conservation of structure (excluding thickness of metallic part of the implantable PROSTHESIS) complicated manufacturing technology and preparation for implantation and implantation of the implantable valve device in accordance with the U.S. Pat. No. 6,730,118 B2. Concurrently a possibility of over expansion has been excluded independently from final diameter and pressure in the balloon catheter.

In addition, a method for comparative numerical evaluation of resistibility of circular and sinuous circles to the radial load is elaborated.

In addition, the availability in the present invention of formed power couch (additional support stent) expands considerably the possibilities for use of polymeric materials in structure and technology of the said US patent.

In addition, a method of facilitating of blood ejection from the left ventricle to the aorta has been suggested (the same method may be also used for different modifications of aortic bioprosthesis', including bypass grafts, and for another cardiovascular devices). The elaborated innovations eliminate the drawbacks of all percutaneous valve devices, wherein take place problems with insufficient volume radial resistance, with unnecessary mass of a carrying frame, with insufficient fixation in the longitudinal direction of the valve device and also with its wear and tear (for example, U.S. Pat. Nos. 5,411,552, 6,168,614 B1, PCT patent application of WO 98/29057 etc.).

1. An Implantable Prosthetic Valve Device, including an additional support stent with transverse sinuous elements, with thrusting angles of the elements of ˜90°, the additional support stent is executed with interior diameter larger than external diameter of the valve prosthesis device after crimping, whereas the sum length of all parts for each of the transverse sinuous element form circle upon expansion until nominal size of implantation in such a way that thrusting angles of elements become equal to 180° or close to that value.

2. An Implantable Prosthetic Valve Device of claim 1, wherein each transverse sinuous element of an additional support stent is executed with unequal thrusting angles of elements, whereas for each transverse sinuous elements of their thrusting angles are reduced from interior to the rims of the additional support stent in such a way that upon expansion the sequence of the circles of nominal sizes created an envelope surface similar to hyperboloid.

3. An Implantable Prosthetic Valve Device of claims 1, wherein a crimping of an additional support stent is executed on the external surface of the crimped valve prosthesis device.

4. An Implantable Prosthetic Valve Device of claim 1, wherein an additional support stent is executed from the same materials as a support stent of the valve prosthesis device.

5. An Implantable Prosthetic Valve Device of claim 1, wherein an additional support stent is executed from Nitinol.

6. An Implantable Prosthetic Valve Device of claim 1, wherein an additional support stent with transverse sinuous elements, with thrusting angles of the elements of 90° is executed from tubes of 13.5 mm-18.0 mm diameters in accordance with nominal sizes of the valve prosthesis device of 19 mm-25 mm diameters.

7. An Implantable Prosthetic Valve Device of claim 1, wherein an additional support stent with transverse sinuous elements, with thrusting angles of the elements of 90°, with nominal size of the valve prosthesis device of 23 mm diameter is executed from the tube of 16.5 mm.

8. An Implantable Prosthetic Valve Device of claims 1, wherein an additional support stent with transverse sinuous elements, with thrusting angles of the elements of 90°, with nominal size of the valve prosthesis device of 19 mm diameter is executed from the tube of 13.5 mm diameter.

9. An Implantable Prosthetic Valve Device of claim 1, wherein an additional support stent with transverse sinuous elements, with thrusting angles of the elements of 90°, with nominal size of the valve prosthesis device of 25 mm diameter is executed from the tube of 18 mm diameter.

10. An Implantable Prosthetic Valve Device of claim 1, wherein an additional support stent is executed from tubes of 12 mm-20 mm diameters, whereas thrusting angles of parts of transverse sinuous elements are chosen in such a way that upon expansion until nominal size of implantation the transverse sinuous elements form a circular structure with thrusting angles of elements of 180°-170°.

11. An Implantable Prosthetic Valve Device of claim 1, wherein an additional support stent is executed with minimal possible thickness of the tube, resistance of which sustains a thrusting force of a balloon catheter.

12. An Implantable Prosthetic Valve Device of claim 1, wherein an additional support stent with ˜0.05 mm-0.10 mm thickness of the tube is executed for combining with a valve prosthesis device with ˜0.25 mm thickness of the tube of the support stent.

13. An Implantable Prosthetic Valve Device of claim 1, wherein an additional support stent is executed with greater amount of transverse sinuous elements.

14. An Implantable Prosthetic Valve Device of claims 1, wherein an additional support stent is executed with greater amount of longitudinal beams.

15. An Implantable Prosthetic Valve Device of claim 1, wherein an additional support stent is executed with lesser amount of longitudinal beams.

16. An Implantable Prosthetic Valve Device of claim 1, wherein an additional support stent is executed with two continuous beams, situated opposite to a stent length in one plane, whereas elements of each continuous beam between two neighboring circles of the additional support stent are presented in a form of the bars of equal resistance to bending along them longitudinal axis.

17. An Implantable Prosthetic Valve Device of claim 1, wherein the circles of an additional stent, made independently from valve prosthesis device for various clinical applications, disconnected and combined along the stent length in one spiral line with one or several threads, whereas instead of two continuous beams, situated opposite to the stent length in one plane, preserve one only.

18. An Implantable Prosthesis Valve Device of claim 16 wherein an additional support stent is executed independently from valve prosthesis device and set up in a blood-vessel, near the side of stent implantation a turbo-germ is being additionally placed, the turbo-germ comprises a thin-walled non-flat diametric partition, which is stretched along the axis of the vessel in the magnitude of its diameter, whereas the concave surfaces of the partition are two fragments of a double-threaded spiral surface with axis, correlating with vessel axis, and inclination angle of the spiral line of about 30°, but each one of the said two fragments is limited by contact area of the spiral line with cylindrical inner wall of the vessel and its axial line.

19. An Implantable Prosthetic Valve Device of claim 16, wherein an additional support stent is executed independently from the prosthesis and set up in a blood-vessel, near the site of stent implantation a turbo-germ is being additionally placed, the turbo-germ comprises a thin-walled diametric partition, made from flat metallic plate with thickness of no more than 0.1 mm and stretched along the axis of the vessel in the magnitude of its diameter, whereas the edges of the partition contact with cylindrical inner wall of the vessel are unfolded symmetrically relative to the vessel axis and each other on the angle of about 60° for imitation of two fragments of a double-threaded spiral surface, but the longitudinal length of the partition near the vessel axis is lessened for easier turn-around of its extreme parts on the said angle of about 60°.

20. An Implantable Prosthetic Valve Device of claim 1, wherein a circular structure of an additional support stent is formed without limitation of a size of the circles diameters.

21. An Implantable Prosthetic Valve Device of claim 1, wherein an additional support stent is executed with length longer than the length of the support stent of the valve prosthesis device, whereas the length of the additional support stent is increased in such a way that one from its parts is inside the descending aorta.

22. An implantable Prosthetic Valve Device of claim 16, wherein an additional support stent, made in individual form for various clinical applications, is executed with length according to the size of an annulus of aortic heart valve for later precise positioning of the valve prosthesis device.

23. An Implantable Prosthetic Valve Device of claim 1, wherein an additional support stent is provided with heavy metal markets so as to enable tracking and determining the valve prosthesis device position.
